(19) United States
(12) Patent Application Publication
(10) Pub. No.: US 2024/0376034 A1
(43) Pub. Date: Nov. 14, 2024

(54) ISOLATION OF DIALKYLENE PHENOLIC GLYCOL ETHER

(71) Applicant: Dow Global Technologies LLC, Midland, MI (US)

(72) Inventor: Edward D. Daugs, Midland, MI (US)

(73) Assignee: Dow Global Technologies LLC, Midland, MI (US)

(21) Appl. No.: 18/686,783

(22) PCT Filed: Aug. 24, 2022
(86) PCT No.: PCT/US2022/041326

§ 371 Date: Feb. 26, 2024

Related U.S. Application Data
(60) Provisional application No. 63/237,173, filed on Aug. 26, 2021.

(51) Int. Cl. C07C 41/40

(52) U.S. Cl. CPC C07C 41/40

(57) ABSTRACT

The present disclosure provides a method for isolating dialkylene phenolic glycol ether (DPGE) from a mixture that includes DPGE and glycosylated phenol impurities. The method includes adding a source of an alkali metal to the mixture to form a phenolic glycol product having an alkali phenolic salt formed from a reaction between the alkali metal and the glycosylated phenol impurities; and separating the DPGE from the alkali phenolic salt in the phenolic glycol product through a thin film evaporation process to produce a dialkylene phenolic glycol ether product. The disclosure also includes a phenolic glycol product that includes DPGE; water; glycosylated phenol impurities; a source of alkali metal; and an alkali phenolic salt formed from a reaction between the alkali metal and the glycosylated phenol impurities.

### FIELD OF DISCLOSURE

Embodiments of the present disclosure are directed towards a process for the isolation of a chemical compound, more specifically, to a process for the isolation of dialkylene phenolic glycol ether.

### BACKGROUND

Producing phenolic glycol ethers (also known as alkylene phenolic glycol ethers) is a well-known process. Examples of such phenolic glycol ethers includes propylene glycol phenyl ether (PPh) and ethylene glycol phenyl ether (EPh). A common technique for producing PPh and/or EPh involves a process in which propylene oxide (PO) and/or ethylene oxide (EO) reacts with phenol in the presence of sodium hydroxide (NaOH) which serves as a catalyst. An example of such a process is described in U.S. Pat. No. 8,558,029.

The reaction conditions used in producing either PPh or EPh can also lead to the production of higher homolog products, e.g., diethylene glycol phenyl ether or dipropylene glycol phenyl ether, among others. The reaction conditions can also lead to the production of phenol-based impurities, which need to be separated from the higher homolog products to produce a marketable product. The presence of these impurities at greater than 1 weight percent (wt. %) in the isolated higher homolog products results in failure to meet product quality specifications for these products. Examples of such impurities include isomeric compounds, such as 2-hydroxyphenylethanol (2-HPEA) and 4-hydroxyphenylethanol (4-HPEA), which can be present with the higher homolog products in amounts of 10 wt. % to 15 wt. %.

In production, a first distillation process separates the PPh or EPh from the higher homolog products and the impurities, and then a second distillation process separates the higher homolog products from the impurities. Attempts to use this second distillation process to separate the higher homolog products from the impurities to produce a marketable product has been, however, very difficult. One reason for this difficulty is the fact that the boiling points of the higher homolog products and many of the impurities are almost identical such that the impurities are co-distilled with the desired higher homolog products.

Therefore, there is a need in the art for a process that can effectively separate these higher homolog products from these impurities to produce a product having less than 1 wt. % impurities to meet product quality specifications for the higher homolog product.

### SUMMARY

The present disclosure provides for a process that can effectively separate higher homolog products, such as diethylene glycol phenyl ether (DiEPh) or dipropylene glycol phenyl ether (DiPPh), from glycosylated phenol impurities, such as 2-hydroxyphenylethanol (2-HPEA) and 4-hydroxyphenylethanol (4-HPEA), to produce a higher homolog product having less than 1 weight percent (wt. %) impurities to meet product quality specifications for the higher homolog product.

The present disclosure provides a method for isolating dialkylene phenolic glycol ether from a mixture that includes dialkylene phenolic glycol ether and glycosylated phenol impurities. The method includes adding a source of an alkali metal to the mixture to form a phenolic glycol product having an alkali phenolic salt formed from a reaction between the alkali metal and the glycosylated phenol impurities; and then separating the dialkylene phenolic glycol ether from the alkali phenolic salt in the phenolic glycol product through a thin film evaporation process to produce a dialkylene phenolic glycol ether product having less than 1 wt. % of the glycosylated phenol impurities based on the total weight of the dialkylene phenolic glycol ether product.

As discussed herein, the boiling points of the higher homolog products, such as the dialkylene phenolic glycol ether, and many of the impurities, such as the glycosylated phenol impurities, in the mixture are almost identical, which makes their separation difficult. For the various embodiments, the glycosylated phenol impurities provided herein have a boiling point at a predetermined pressure that are approximately equivalent to the boiling point of the dialkylene phenolic glycol ether at the predetermined pressure. This is the case in which the dialkylene phenolic glycol ether is diethylene glycol phenyl ether and the glycosylated phenol impurities include 2-hydroxyphenylethanol and 4-hydroxyphenylethanol. Similarly, this is the case in which the dialkylene phenolic glycol ether is dipropylene glycol phenyl ether and the glycosylated phenol impurities include 2-hydroxyphenylpropanol and 4-hydroxyphenylpropanol.

For the various embodiments, adding the source of the alkali metal to the mixture to form the phenolic glycol product effectively lowers the vapor pressure of the glycosylated phenol impurities as they are converted to the alkali phenolic salt in the reaction with the alkali metal. With this change in vapor pressure for the glycosylated phenol impurities in the phenolic glycol product, the dialkylene phenolic glycol ether can more easily be separated where it had at one time been very difficult.

For the various embodiments, the alkali metal used in the present method is selected from the group consisting of sodium and potassium. Sources of these alkali metals can include alkali hydrides. The alkali hydride can be selected from the group consisting of sodium hydride and potassium hydride. In an alternative embodiment, the source for the alkali metal can include an alkali hydroxide. The alkali hydroxide can be selected from the group consisting of sodium hydroxide and potassium hydroxide.

In addition, the source for the alkali metal can be an aqueous solution of the alkali hydroxide. In a specific example, the aqueous solution of the alkali hydroxide is an aqueous sodium hydroxide solution having an amount of sodium hydroxide up to 50 percent by weight of the aqueous sodium hydroxide solution. As water can be present in the mixture, the phenolic glycol product can undergo a dehydrating step to remove at least a portion of the water from the phenolic glycol product prior to separating the dialkylene phenolic glycol ether from the alkali phenolic salt in the phenolic glycol product.

For the various embodiments, adding the source of the alkali metal to the mixture can include adding 1.6 wt. % to 5 wt. % of the alkali metal hydroxide to the mixture, where the wt. % is based on the total weight of the phenolic glycol product. In an alternative embodiment, adding the source of the alkali metal to the mixture can include adding 2 wt. % to 3.5 wt. % of the alkali metal hydroxide to the mixture, wherein the wt. % is based on the total weight of the phenolic glycol product.

The present disclosure also provides for a phenolic glycol product that includes dialkylene phenolic glycol ether; water; glycosylated phenol impurities; a source of alkali metal; and an alkali phenolic salt formed from a reaction between the alkali metal and the glycosylated phenol impurities, where the phenolic glycol product has less than 1 wt. % of the glycosylated phenol impurities based on the total weight of the dialkylene phenolic glycol ether product. As discussed herein, the source of alkali metal can be selected from the group consisting of sodium, sodium hydroxide, sodium hydride, potassium, potassium hydroxide and potassium hydride.

For the various embodiments, the phenolic glycol product can include 1.6 wt. % to 5 wt. % of the source of alkali metal, the wt. % based on the total weight of the phenolic glycol product. In an alternative embodiment, the phenolic glycol product can include 2 wt. % to 3.5 wt. % of the source of alkali metal, the wt. % based on the total weight of the phenolic glycol product. For the phenolic glycol product, the dialkylene phenolic glycol ether can be diethylene glycol phenyl ether and the glycosylated phenol impurities can include 2-hydroxyphenylethanol and 4-hydroxyphenylethanol. In an alternative embodiment, the dialkylene phenolic glycol ether in the phenolic glycol product is dipropylene glycol phenyl ether and the glycosylated phenol impurities include 2-hydroxyphenylpropanol and 4-hydroxyphenylpropanol.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF DRAWINGS

FIG. 1 is an illustration of a laboratory rolled film evaporator for use according to the present disclosure.

FIG. 2 is a graph demonstrating the relationship of the gas chromatography area percent data for 2-hydroxyphenylethanol in an overhead product as a function of the weight percent of NaOH present in a test sample of EPh Basic being separated in the laboratory rolled film evaporator of FIG. 1.

### DETAILED DESCRIPTION

The numerical ranges in this disclosure are approximate, and thus may include values outside of the range unless otherwise indicated. Numerical ranges include all values from and including the lower and the upper values, in increments of one unit, provided there is a separation of at least two units between any lower value and any higher value. As an example, if a compositional, physical or other property, such as, for example, molecular weight, viscosity, melt index, etc., is from 100 to 1,000, it is intended that all individual values, such as 100, 101, 102, etc., and sub-ranges, such as 100 to 144, 155 to 170, 197 to 200, etc., are expressly enumerated. For ranges containing values which are less than one or containing fractional numbers greater than one (e.g., 1.1, 1.5, etc.), one unit is 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, the relative amount of oxide to phenol, the relative amount of catalyst in the reaction mass, and various temperature and other process parameters.

“Catalytic amount” means the amount necessary to promote the reaction of a phenolic compound and an alkylene oxide under reactive conditions to form phenolic glycol ether at a detectable level, preferably at a commercially acceptable level. If a catalyst is used, then typically, the minimum amount of catalyst is at least 100 parts per million (ppm). For the various embodiments, the amount of catalyst, when used, can range from 100 ppm to 4,000 ppm.

“Homogeneous catalyst” and like terms means a catalyst that is dispersed, preferably uniformly, through the phenolic compound or reaction mass as opposed to, for example, a catalyst bound to an ion exchange resin or a fixed-bed catalyst.

“Basic homogeneous catalyst” and like terms means a homogenous catalyst that in aqueous solution having a pH greater than 7.

“Acidic homogeneous catalyst” and like terms means a homogeneous catalyst that in aqueous solution having a pH of less than 7.

“Isothermal reactive conditions”, “isothermal reactor”, “isothermal reaction zone”, “isothermal reaction” and like terms mean reactive conditions in which the temperature is held constant, or the temperature of the reactor or zone is held constant, or a chemical reaction proceeds to completion at one temperature, i.e., a change in temperature is not necessary for the reaction to continue to completion.

“Adiabatic reactive conditions”, “adiabatic reactor”, “adiabatic reaction zone”, adiabatic reaction” and like terms mean reactive conditions, or a reactor or zone, or a reaction in which little, if any, loss or gain of heat from external sources occurs or is experienced.

“First intermediate phenolic glycol ether product” and like terms means the product that is produced from the reaction of a phenolic compound with an alkylene oxide in an isothermal reactor or reaction zone. This product includes not only phenolic glycol ether and dialkylene phenolic glycol ether, but also catalyst, unreacted phenolic compound and alkylene oxide, water and byproducts (e.g., glycosylated phenol impurities).

“Second intermediate phenolic glycol ether product” and like terms means the product that is produced from the reaction of a phenolic compound with an alkylene oxide in an adiabatic reactor or reaction zone. This product includes all the components of the first intermediate phenolic glycol ether product but at different compositional ratios, e.g., it contains more phenolic glycol ether and less unreacted alkylene oxide and phenolic compound.

“Reaction mass”, “reacting system” and like terms means the combination of materials necessary or ancillary to a reaction, typically under reactive conditions. Depending upon the moment in time in which the reaction mass is characterized, it will or can contain the reactants, catalyst, solvent, products, byproducts, impurities, and the like. The typical reaction mass that forms a part of this disclosure after the reaction has begun will include unreacted alkylene oxide and phenolic compound, an alkali metal hydroxide, phenolic glycol ether, dialkylene phenolic glycol ether, byproduct glycols (e.g., glycosylated phenol impurities) and water.

“Nonaqueous process” and like terms means in the context of this disclosure that the reaction mass contains little, if any, water. In the process of this disclosure, the only water intentionally introduced into the reaction mass is that necessary to dissolve and assist in the dispersion of the catalyst. Any other water present is either a byproduct of the reaction chemistry or as an impurity associated with one of the reactants. The total amount of water in the second intermediate phenolic glycol ether product typically does not exceed 1 weight percent (wt. %), preferably does not exceed 0.5 wt. % and more preferably does not exceed 0.05 wt. % (500 ppm) based on the weight of the second intermediate phenolic glycol ether product.

“Continuous process” and like terms means that the process is operated at a steady state, i.e., the reactants are fed to the reactor or reaction zone at a rate substantially in balance with the rate that product is removed from the reactor or reaction zone such that the reaction mass in the reactor or reaction zone is relatively constant in volume and composition. Continuous process does not include a batch or semi-batch process, the former characterized by a depletion of reactants and a growth of product over time, and the latter typically characterized by the unbalanced addition of reactant and removal of product over time.

Phenols, sometimes called phenolics, are a class of organic compounds consisting of a hydroxyl group (—OH) attached to an aromatic hydrocarbon group. The simplest of the class is phenol (C₆H₅OH). The phenolic compounds that can be used in the practice of this disclosure are typically monovalent and include phenol: phenols having a hydrocarbon substituent such as o-, m- or p-cresol, o-, m- or p-ethylphenol, o-, m- or p-t-butylphenol, o-, m-, or p-octylphenol, 2,3-xylenol, 2,6-xylenol, 3,4-xylenol, 3-5-xylenol, 2,4-di-t-butylphenol; phenols having a substituent group such as an aromatic substituent or an aromatic ring e.g., o-, m- or p-phenylphenol, p-alpha-cumyl)phenol, and 4-phenoxyphenol; phenols having an aldehyde group such as o-, m- or p-hydroxybenzaldehyde; phenols having a substituent group with an ether linkage such as guaiacol and guaethol; phenols having a substituent group such as a hydroxyl group with a property inherent to alcohol (hereinafter, called as “alcoholic hydroxyl group”) e.g., p-hydroxyphenethyl alcohol; phenols having a substituent group with an ester linkage such as p-hydroxy benzoic methyl, p-hydroxyphenylacetic acid methyl ester, and heptylparaben; and phenols having a halogen group such as 2,4,6-trichlorophenol. Among these, phenol and cresol are preferred. These phenols may be used alone or in any combination with one another.

The alkylene oxides (also known as epoxides) that can be used in the practice of this disclosure include ethylene oxide, propylene oxide, isobutylene oxide, 1,2-butylene oxide, 2,3-butylene oxide, and pentylene oxide; aromatic alkylene oxides such as stylene oxide; and cyclohexane oxide. These alkylene oxides may be used alone or in any combination with one another. Among the alkylene oxide compounds, preferred are aliphatic alkylene oxides having 2 to 4 carbon atoms such as ethylene oxide, propylene oxide, isobutylene oxide, and 2,3-butylene oxide. Although the alkylene oxide is typically added as a liquid, it can be added as a gas.

While the catalyst used in the practice of this disclosure can be any appropriate acid or base, e.g., a Lewis acid or base, preferably the catalyst is a base. Alkaline materials effective for catalyst generation include alkali metals, alkali hydroxides, alkali hydrides, and carbonates, alkaline earth metal hydroxides, tetra-alkyl ammonium hydroxide and organic bases (e.g., pyridine, trimethyl amine and imidazole). The preferred alkali metals catalysts are sodium and potassium. The preferred alkali hydride catalysts are sodium hydride and potassium hydride. The preferred alkali hydroxides catalysts are sodium hydroxide and potassium hydroxide. The catalyst, can be added neat, usually dissolved in a small amount of water, or formed in situ. The catalyst is used in a homogeneous manner, i.e., it is dispersed, preferably uniformly, through the reaction mass. Typically, the catalyst is mixed with the phenolic compound before the phenolic compound is mixed with the alkylene oxide.

The process provided in the present disclosure is particularly useful for the production of ethylene glycol phenyl ether (EPh) and/or propylene glycol phenyl ether (PPh) from phenol, ethylene oxide and/or propylene oxide, and sodium or potassium hydroxide catalyst. By use of a large excess of phenol, minimal, if any, water and a homogeneous catalyst, EPh and PPh are selectively made with a minimum production of glycol byproducts.

For the methods provided in this disclosure, the phenolic compound, alkylene oxide and catalyst are continuously added in any conventional manner to an isothermal reactor or the isothermal zone of a multizone reactor. The phenolic compound is added in excess relative to the alkylene oxide and as noted above, the catalyst is often pre-mixed with the phenolic compound, e.g., as part of a phenolic recycle stream, before the alkylene oxide is mixed with the phenolic compound, etc. The size of the excess amount of phenolic compound can and will vary with the desired operation of the process and product mix. Typically, the more phenol present the faster the reaction proceeds and the fewer by-products are made. The phenolic compound is typically present in a stoichiometric molar excess ranging from as little as 0.5% to as much as 100% or even 200% excess phenolic compound relative the alkylene oxide.

The phenolic compound, alkylene oxide and catalyst are contacted with one another in the isothermal reactor or zone under isothermal reactive conditions. These conditions include a temperature between ambient (e.g., 23 degrees Celsius (° C.)) and 200° C., preferably between 100° C. and 180° C. and more preferably from 120° C. to 170° C.; and a pressure between 8,000 and 50,000 millimeters of mercury (mmHg, or between 1.067 and 6.667 megaPascal (MPa)), preferably between 20,000 and 40,000 mmHg (2.067 and 5.333 MPa) and more preferably between 25,000 and 35,000 mmHg (3.333 and 4.666 MPa). The reaction mass in the isothermal reactor or zone is essentially free of water except for that used to dissolve the catalyst or that formed as a byproduct or introduced as an impurity, and it is subject to agitation by any conventional means, e.g., stirring, turbulent flow, etc. The reaction mass is resident in the isothermal reactor or zone until a majority of the alkylene oxide is converted thus forming a first intermediate phenolic glycol ether product, and then this product is transferred by known means to an adiabatic reactor or zone in which essentially all of the remaining alkylene oxide is converted to form the second intermediate phenolic glycol ether product.

The isothermal and adiabatic reaction zones can be reactors that are separate and distinct from one another and connected in series, or they can be zones within a single reactor structure. For example, the isothermal reactor can be a coiled reactor consisting of multiple spiral parallel coils of varying number (e.g., 2-4 coils) within a boiling bath all contained in a metal shell. The heat of reaction is removed via boiling water on the shell side of the coils while the reactive process occurs within the coils themselves. The adiabatic section can be volume designed (whether it is insulated piping or an insulted process vessel) to provide sufficient residence for complete oxide conversion. The temperature of the first reactor zone may be different, and is typically lower, than the temperature of the second reactor zone. This temperature difference is typically from 0 to 40° C., more typically from 0 to 20° C. and even more typically from 0 to 10° C. Other than this temperature difference, the adiabatic reactive conditions of the adiabatic reactor or zone are essentially the same as the isothermal reactive conditions of the isothermal reactor or zone. The temperature of the first intermediate phenolic glycol ether product typically may be adjusted to the temperature of the adiabatic reactor or zone by passing through one or more heat exchangers as it moves from the isothermal reactor or zone to the adiabatic reactor or zone. Once the conversion of the alkylene oxide in the adiabatic reactor or reaction zone is complete, the second intermediate phenolic glycol ether product is discharged and subjected to a purification operation, which includes the method of the present disclosure.

According to the present disclosure, the second intermediate phenolic glycol ether product is transferred to a separation station or zone, e.g., a first distillation column, in which the unreacted phenolic compound, catalyst and water are recovered and the remainder of the second intermediate phenolic glycol ether product is transferred to one or more additional distillation columns in which the phenolic glycol ether is recovered in high purity, typically greater than 95 wt. %, preferably greater than 99 wt. % and more preferably greater than 99.5, wt. % pure. In the second column (and additional columns if used), the purified phenolic glycol ether is recovered as a side-draw stream, and the overhead stream containing the remainder of the stream is returned to the first column to recover additional unreacted phenolic compound not recovered during the first pass.

The recovered phenolic compound and residual water stream from the first distillation column is then typically transferred to a drying station at which it is subjected to a drying operation. An example of a drying station is a multi-stage distillation column in which water (the lighter component) is physically separated from phenol (the heavier component) by adjusting the temperature and pressure profile in the column. Fresh phenol and/or catalyst can be added before the drying column to remove any water present in these materials. The resulting phenol and catalyst mixture is removed and returned to the reactor system. Other drying methods that can be used include mole-sieve, desiccant, and membrane.

In addition to recovering the purified phenolic glycol ether from the second column, a mixture that includes the higher homolog products, e.g., dialkylene glycol phenyl ether and trialkylene glycol ether, among others, along with phenol-based impurities produced during the production of the phenolic glycol ether product is recovered as a bottoms product from the second distillation column. When ethylene oxide is used as a reactant in forming the phenolic glycol ether, examples of such impurities include isomeric compounds, such as 2-hydroxyphenylethanol (2-HPEA) and 4-hydroxyphenylethanol (4-HPEA), which can be present with the higher homolog products (e.g., diethylene glycol phenyl ether) in amounts of 10 weight percent (wt. %) to 15 wt. %, based on the total weight of the mixture.

As discussed herein, the reaction conditions used in producing the phenolic glycol ether product (e.g., ethylene glycol phenyl ether (EPh) or propylene glycol phenyl ether (PPh)) can also lead to the production of higher homolog products, e.g., dialkylene phenolic glycol ethers such as diethylene glycol phenyl ether or dipropylene glycol phenyl ether, among others, along with glycosylated phenol impurities, which need to be separated from the higher homolog products in order to produce a marketable dialkylene phenolic glycol ether product. Examples of such glycosylated phenol impurities include isomeric compounds, such as 2-HPEA and 4-HPEA, noted herein.

According to the present disclosure, the method for isolating the dialkylene phenolic glycol ether from the mixture that includes the glycosylated phenol impurities involves adding a source of an alkali metal to the mixture to form a phenolic glycol product having an alkali phenolic salt formed from a reaction between the alkali metal and the glycosylated phenol impurities. As provided herein, many of the glycosylated phenol impurities have a boiling point at a predetermined pressure that are approximately equivalent to the boiling point of the dialkylene phenolic glycol ether at the predetermined pressure. As a result, in using a traditional distillation process the glycosylated phenol impurities would co-distil with the desired higher homolog products.

The present disclosure, however, reacts the glycosylated phenol impurities with the alkali metal to form the alkali phenolic salt in the phenolic glycol product. For the various embodiments, adding the source of the alkali metal to the mixture to form the phenolic glycol product effectively lowers the vapor pressure of the glycosylated phenol impurities as they are converted to the alkali phenolic salt in the reaction with the alkali metal. With this change in vapor pressure for the glycosylated phenol impurities in the phenolic glycol product, the dialkylene phenolic glycol ether can more easily be separated where it had at one time been very difficult. So, for the various embodiments, this conversion to the alkali phenolic salt to change (e.g., lower) the vapor pressure of the glycosylated phenol impurities allows for the separation of the dialkylene phenolic glycol ether from the alkali phenolic salt in the phenolic glycol product through a thin film evaporation process to produce the dialkylene phenolic glycol ether product having less than 1 wt. % of the glycosylated phenol impurities based on the total weight of the dialkylene phenolic glycol ether product.

For the various embodiments, adding the source of the alkali metal to the mixture to form the phenolic glycol product having the alkali phenolic salt can be done at a temperature and pressure in a range of 0 degrees Celsius (° C.) to 200° C. and 10 kilopascal (kPa) to 200 kPa. Preferably, adding the source of the alkali metal to the mixture to form the phenolic glycol product is done at a temperature of 0° C. to 50° C. and a pressure of 50 kPa to 150 kPa, and more preferably at a temperature of 17° C. to 27° C. and a pressure of 90 kPa to 110 kPa and most preferably at a temperature of 25° C. and a pressure of 101 kPa. The mixture and the source of the alkali metal can be mixed using known mixing techniques, such as through mechanical stirring, to facilitate the reaction to form the phenolic glycol product at the desired temperature and pressure.

For the various embodiments, a thin film evaporation process is used to separate the dialkylene phenolic glycol ether from the alkali phenolic salt in the phenolic glycol product to produce a dialkylene phenolic glycol ether product having less than 1 wt. % of the glycosylated phenol impurities based on the total weight of the dialkylene phenolic glycol ether product. As provided herein, an example of the dialkylene phenolic glycol ether is diethylene glycol phenyl ether, where the glycosylated phenol impurities include 2-hydroxyphenylethanol and 4-hydroxyphenylethanol. In an alternative example, the dialkylene phenolic glycol ether is dipropylene glycol phenyl ether, where the glycosylated phenol impurities include 2-hydroxyphenylpropanol and 4-hydroxyphenylpropanol.

The thin film evaporation process of the present disclosure is a separation technique that utilizes temperature and pressure variables to separate components based at least in part on their vapor pressure at a given temperature and pressure. For the various embodiments, the thin film evaporation process may also be known as and/or take the form of a thin film evaporator, a wiped film evaporator, a rolled film evaporator, a falling film evaporation or a climbing film evaporator. The thin film evaporation process is preferred as this process allows for the separation of heat sensitive components, such as the dialkylene phenolic glycol ether present in the mixture with the glycosylated phenol impurities.

For the present embodiments, the thin film evaporation process can be carried out at a vacuum sufficiently low enough to allow the thin film evaporator to operate at a temperature of about 210° C. or below. For example, the thin film evaporation process can be carried out at a pressure of 1.3 kPa or below at a temperature of about 210° C. or below; preferably, the thin film evaporation process can be carried out at a pressure of 670 Pa or below at a temperature of about 210° C. or below; and most preferably the thin film evaporation process can be carried out at a pressure of 133 Pa or below at a temperature of about 210° C. or below. In more specific examples, thin film evaporation process can be carried out at a temperature of 0° C. to 210° C. and a pressure of 0.1 Pa to 1.3 kPa and more preferably at a temperature of 100° C. to 210° C. and a pressure of 10 Pa to 700 Pa. The residence time for the separation of the dialkylene phenolic glycol ether from the alkali phenolic salt in the phenolic glycol product using the thin film evaporation process can be on the order of 0.5 seconds to 5 minutes. Other times are also possible.

For the various embodiments, the alkali metal used in the present method is selected from the group consisting of sodium, potassium, lithium, and calcium. Preferably, the alkali metal used in the present method is selected from the group consisting of sodium and potassium. Sources of these alkali metal can include an alkali hydride. The alkali hydride can be selected from the group consisting of sodium hydride, potassium hydride, calcium hydride and lithium hydride. Preferably, the alkali hydride can be selected from the group consisting of sodium hydride and potassium hydride. In an alternative embodiment, the source for the alkali metal can include an alkali hydroxide. The alkali hydroxide can be selected from the group consisting of sodium hydroxide, potassium hydroxide, calcium hydroxide and lithium hydroxide. Preferably, the alkali hydroxide can be selected from the group consisting of sodium hydroxide and potassium hydroxide.

Preferably, the source for the alkali metal is an aqueous solution of the alkali hydroxide, as provided herein. Having an aqueous solution of the alkali hydroxide provides a soluble form of the alkali metal that can mix easily with the mixture that includes the dialkylene phenolic glycol ether and the glycosylated phenol impurities. The aqueous solution can have an alkali hydroxide concentration over a wide range of values, as provided herein, as long as the aqueous solution remains miscible with the mixture that includes the dialkylene phenolic glycol ether and the glycosylated phenol impurities. For the various embodiments, the aqueous solution of the alkali hydroxide can have an amount of the alkali hydroxide from 1 to 50 percent by weight (wt. %) based on the total weight of the aqueous solution. Preferably, the aqueous solution of the alkali hydroxide can have an amount of the alkali hydroxide from 30 to 50 wt. % based on the total weight of the aqueous solution.

For the various embodiments, it is also appreciated that the amount of water added with the source of the alkali metal to the mixture to form the phenolic glycol product may need to be removed prior to separating the dialkylene phenolic glycol ether from the alkali phenolic salt in the phenolic glycol product through the thin film evaporation process. As such, when using the aqueous solution of the alkali hydroxide it is preferred to have concentrations of the alkali hydroxide at or closer to the 50 wt. % based on the total weight of the aqueous solution. While it is possible to use an aqueous solution of the alkali hydroxide having a concentration of the alkali hydroxide greater than 50 wt. % based on the total weight of the aqueous solution, there may be issues with viscosity of the resulting mixture that are not overly desired. In a specific example, the aqueous solution of the alkali hydroxide is an aqueous sodium hydroxide solution having an amount of sodium hydroxide up to 50 wt. % based on the total weight of aqueous sodium hydroxide solution. In a more specific example, the aqueous solution of the alkali hydroxide is an aqueous sodium hydroxide solution having 50 wt. % sodium hydroxide based on the total weight of aqueous sodium hydroxide solution.

For the various embodiments, when the mixture includes water that needs to be removed a variety of dehydrating steps can be used to remove at least a portion of the water from the phenolic glycol product prior to separating the dialkylene phenolic glycol ether from the alkali phenolic salt in the phenolic glycol product. Such processes can include an evaporation process that removes the water from the phenolic glycol product by adding energy (e.g., heat) at a predetermined pressure to change the physical state of the water from a liquid, present in the phenolic glycol product, to a gas, which can be condensed so as to remove the water from the phenolic glycol product, Examples of temperatures and pressures for the evaporation process include a temperature of 50 to 110° C. and a pressure of 12.3 kPa to 101.3 kPa. Upon removing water, the dialkylene phenolic glycol ether is separated from the alkali phenolic salt in the phenolic glycol product through the thin film evaporation process as provided herein, where the residual amount of water, if any, that remains in the phenolic glycol product helps to determine the rate at which the phenolic glycol product can be fed to the thin film evaporator while maintaining the required vacuum.

For the various embodiments, the amount of impurities, such as the glycosylated phenol impurities, formed in producing the phenolic glycol ether can vary depending upon the reaction conditions and the relative amounts of the phenolic compound and the alkylene oxide, as discussed herein. As a result, the amount of the alkali metal in the source of the alkali metal added to the mixture to form the phenolic glycol product will increase or decrease according to the amount of impurities in the mixture. The amount of impurities produced in such reactions can, however, be measured using known techniques, where such information can then used in determining the amount of the alkali metal to add to the mixture to form the phenolic glycol product. For example, a molar amount of the alkali metal in the source of the alkali metal that is added to the mixture to form the phenolic glycol product can be matched to the molar amount of the impurities measured in the mixture. So, the molar amount of the alkali metal in the source of the alkali metal will increase or decrease according to the measured amount of impurities measured in the mixture.

Based on the above discussion, the source of the alkali metal added to the mixture can be in a molar amount ratio of 0.5:1 moles of alkali metal:moles of impurities to 1.1:1 moles of alkali metal:moles of impurities. Other ratios for such molar amounts can range from 0.75:1 moles of alkali metal:moles of impurities to 1:1 moles of alkali metal:moles of impurities or 0.8:1 moles of alkali metal:moles of impurities to 1:0.9 moles of alkali metal:moles of impurities. In an alternative approach, adding the source of the alkali metal to the mixture can include adding 1.6 wt. % to 5 wt. % of the alkali metal hydroxide to the mixture, where the wt. % is based on the total weight of the phenolic glycol product. In an alternative embodiment, adding the source of the alkali metal to the mixture can include adding 2 wt. % to 3.5 wt. % of the alkali metal hydroxide to the mixture, wherein the wt. % is based on the total weight of the phenolic glycol product.

The present disclosure also provides for the phenolic glycol product, as provided herein, where the phenolic glycol product includes dialkylene phenolic glycol ether; water; glycosylated phenol impurities; a source of alkali metal; and an alkali phenolic salt formed from a reaction between the alkali metal and the glycosylated phenol impurities, where the phenolic glycol product has less than 1 wt. % of the glycosylated phenol impurities based on the total weight of the dialkylene phenolic glycol ether product.

The present disclosure also provides for a phenolic glycol product, as discussed herein. As provided herein, the phenolic glycol product includes dialkylene phenolic glycol ether; water; glycosylated phenol impurities; a source of alkali metal; and an alkali phenolic salt formed from a reaction between the alkali metal and the glycosylated phenol impurities, where the phenolic glycol product has less than 1 wt. % of the glycosylated phenol impurities based on the total weight of the dialkylene phenolic glycol ether product. As discussed herein, the source of alkali metal can be selected from the group consisting of sodium, sodium hydroxide, sodium hydride, potassium, potassium hydroxide and potassium hydride. In the way of a specific example, for the various embodiments, the phenolic glycol product can include 1.6 wt. % to 5 wt. % of the source of alkali metal, the wt. % based on the total weight of the phenolic glycol product. In an alternative embodiment, the phenolic glycol product can include 2 wt. % to 3.5 wt. % of the source of alkali metal, the wt. % based on the total weight of the phenolic glycol product. For the phenolic glycol product, the dialkylene phenolic glycol ether can be diethylene glycol phenyl ether and the glycosylated phenol impurities can include 2-hydroxyphenylethanol and 4-hydroxyphenylethanol. In an alternative embodiment, the dialkylene phenolic glycol ether in the phenolic glycol product is dipropylene glycol phenyl ether and the glycosylated phenol impurities include 2-hydroxyphenylpropanol and 4-hydroxyphenylpropanol.

Although the disclosure has been described in considerable detail by the preceding specification, this detail is for the purpose of illustration and is not to be construed as a limitation upon the following appended claims. All U.S. patents, allowed U.S. patent applications and U.S. Patent Application Publications are incorporated herein by reference.

### EXAMPLES

### Materials/Ingredients

The following Examples (Ex) and Comparative Examples (CE) use what is referred to herein as “EPh Basic.” “EPh Basic” is the second intermediate phenolic glycol ether product produced in accordance with U.S. Pat. No. 8,558,029. “EPh Basic” is a mixture of phenolic glycol ethers, unreacted phenolic compound (e.g., phenol), catalyst (e.g., NaOH), water and byproduct glycol impurities that include glycosylated phenol impurities (e.g, 2-hydroxyphenylethanol (2-HPEA) and 4-hydroxyphenylethanol (4-HPEA)). The phenolic glycol ethers can include ethylene glycol phenyl ether (EPh), diethylene glycol phenyl ether (DiEPh), triethylene glycol phenyl ether (TriEPh) and tetraethylene glycol phenyl ether (TetraEPh).

For the present Examples, the production of EPh Basic is as follows. Charge a 2 liter (L) stainless steel Parr reactor with 396.4 grams (g) of phenol and 0.79 g of solid sodium hydroxide. Seal and pressure check the reactor. Heat the reactor to 160° C. after which add 84.1 g of ethylene oxide over 31 seconds. After five hours, cool and unload the resulting EPh reaction product. Analyze the EPh reaction product using GC analysis as described herein. GC analysis of the EPh reaction product, as provided below, indicates the presence of phenol, EPh, DiEPh, TriEPh, TetraEPh, 2-hydroxyphenylethanol (2-HPEA), 4-hydroxyphenylethanol (4-HPEA) and additional high molecular weight impurities (e.g., highers 1 and highers 2).

To produce the EPh Basic, add 157.4 g of the EPh reaction product to a 250-mL round-bottom flask with magnetic stirring in a temperature-controlled heating mantle and topped with an 18 inch-tall vacuum jacketed distillation column, reflux splitter, and condenser set at 50° C. Place the system under a vacuum of 16 kilopascal (KPa, 120 torr) and heat the EPh reaction product to 160° C. Remove the overhead product at a 5 to 1 reflux ratio until the phenol content of the overhead product drops below 98%. Slowly decrease the vacuum to 8.8 KPa (66 torr) to maintain a temperature in the 250-mL flask of 160-165° C. Continue the distillation to remove an overhead product having a phenol content of greater than 87% and then an overhead product having an EPh content of greater than 92% EPh, after which stop the distillation process. The bottom product from the distillation process is the EPh Basic, which is a mixture containing EPh, DiEPh, TetraEPh, 2-HPEA, 4-HPEA, highers 1, highers 2 and less than 0.2 wt. % phenol. Table 1 provides a gas chromatography (GC) analysis, as described below, of the EPh Basic produced according to the above description.

TABLE 1

| | GC Retention Time | EPh Basic |
|---|---|---|
| Component | Minutes | GC Area % |
| Phenoxyethanol (EPh) | 18.4 | 14.21 |
| 2-HPEA | 21.1 | 9.74 |
| 4-HPEA | 22.2 | 2.29 |
| Phenoxyethoxyethanol | 23.4 | 59.37 |
| (DiEPh) | | |
| Triethyleneglycol Phenyl | 25.6 | 7.66 |
| Ether (TriEPh) | | |
| Highers 2 | 26.9 | 1.71 |
| Highers 1 | 27.3 | 3.62 |
| Tetraethyleneglycol Phenyl | 29.4 | 1.19 |
| Ether (TetraEPh) | | |

50% Aqueous Sodium Hydroxide (Sodium Hydroxide Solution 50% available from Sigma Aldrich).

BSTFA containing 1 wt. % chlorotrimethylsilane reagent (BSTFA Solution, Sigma Aldrich).

### Test Measurements

Base Concentration Measurement—Measure a base concentration using a calibrated Mettler Toledo DL70 Autotitrator by dilution of 0.1 to 5 gram (g) of the sample in aqueous 2-propanol and titrating with 0.0100N hydrochloric acid. Report the result as weight percent (wt. %) NaOH.

Water Concentration Measurement (Karl Fischer Analysis)—Measure water concentrations according to ASTM E203 using a Mettler DL 18 Karl Fischer Water Titrator attached to a Mettler-Toledo GA 42 printer and a Mettler Toledo AE160 analytical balance. Calibrate the titrator using a water standard from Aquastar® containing 1.00 percent by weight water.

Gas Chromatography (GC) Analysis—Use a Hewlett Packard 6890 GC using N,O-Bis(trimethylsilyl)trifluoroacetamide (BSTFA) derivatization for GC Analysis. For the analysis, mix a 200 microliter (μL) of sample and hold for at least fifteen minutes with 0.5 milliliter (mL) of pyridine and 0.5 mL of the BSTFA solution before injecting 4 μL of the sample with a 72 mL/minute (min) split flow (split ratio 20:1) onto a 0.32 millimeter (mm) i.d. 60 meter (m) 1 micrometer (μm) ZB-1 column at 50 degrees Celsius (° C.) and increasing at a rate of 8° C./min to 280° C. The injector temperature is 275° C. and the FID detector temperature is 300° C. Use helium as the carrier gas at a head pressure of 172.4 kilopascal (KPa, 25 pounds per square inch), giving a 3.6 mL/min or 41 centimeter/second (cm/sec) flow. Begin integration after 10 minutes. Table 2 provides the GC Retention Times, Specification Limits, and Typical Product Compositions.

### Experimental Equipment and Procedure

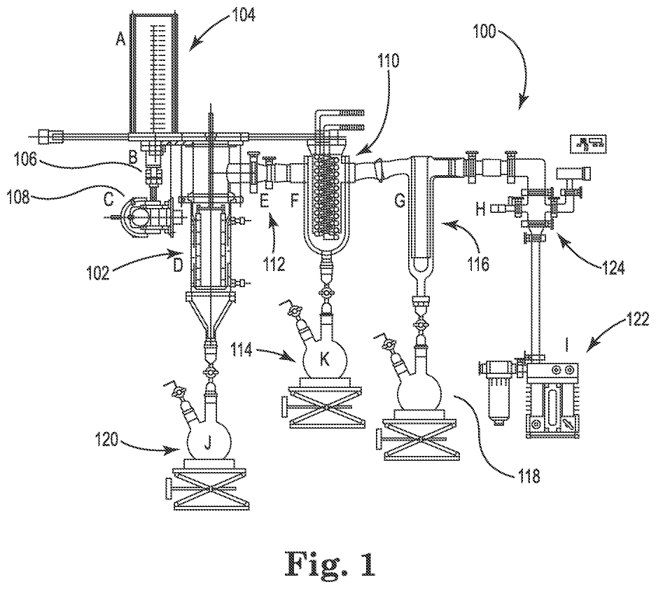

Conduct the experiments on a customized UIC RFT-6 laboratory rolled film evaporator (UIC GmbH, Alzenau-Horstein, Germany). FIG. 1 illustrates the customized UIC RFT-6 laboratory rolled film evaporator 100. The UIC RFT-6 laboratory rolled film evaporator 100 includes an evaporator 102 having an internal stainless steel evaporation area of 0.06 square meters. The internal stainless steel evaporative area of the evaporator 102 is split into an upper zone and a lower zone of equal surface area. Each of the upper zone and the lower zone is independently temperature-controlled via their own Marlotherm® SH heat transfer fluid filled Julabo SE-6 hot oil bath (Julabo USA, Inc.).

For testing, add the amount of the 50% aqueous sodium hydroxide solution to the test sample of the EPh Basic according to Table 1, below. Load the test sample of the EPh Basic into the 2-liter jacketed feed vessel 104 and use a Mahr Feinprüf Model N19 0.6 spinning pump 106 (Mahr-Gruppe) having a feed rate of 0.66 cm³/rotation to meter the test sample into the evaporative area of the evaporator 102. Control the spin rate of the pump 106 with a Bodine Electric Company Model 42A5BEPM-5N Gearmotor 108 attached to a KB Penta-Drive™ Model KBPC-240D Speed Control. Control the temperature in the jacketed feed vessel 104 using an ethylene glycol/water filled Julabo 25 recirculating bath (Julabo USA, Inc.).

During the test, the test sample enters the evaporator 102 of the UIC RFT-6 laboratory rolled film evaporator 100 through a hole near the center of the evaporator area, where it contacts a rotating stainless steel plate at the top of the roller basket and is transferred to the inner walls of the evaporator body to be rolled out into a film by the floating Teflon rollers on three vertical shafts of the wiper basket as it flows down the wall of the evaporator section. Control the wiper basket speed using an IKA RW20 DZMn stirrer motor (IKA Works, Inc.). Use a tap water cooled 0.3 to 0.6 square meter surface area glass condenser 110 to condense the overhead product exiting the evaporator section 112 from the internal evaporative 102. Collect the distillate from the condenser 110 in a round bottom flask 114. Use a dry ice filled cold trap 116 to trap any vapor that fails to condense in the glass condenser 110, collecting any condensate in the round bottom flask 118. Collect bottoms residue from the evaporative area 102 of the rolled film evaporator 100 in the round bottom flask 120 positioned below the evaporator body. Supply the vacuum for the rolled film evaporator 100 using a Leybold TRIVAC® D4B rotary vane vacuum pump 122, controlling the pump 122 with a Leybold EV 016 DOS AB dosing valve 124 supplied with 48.3 kPa (7 PSig) nitrogen.

Table 2 provides the conditions and settings of the rolled film evaporator 100 used in testing both the Examples (Ex) and Comparative Examples (CE), as follows. CE A through CE D do not include the use of the 50% Aqueous Sodium Hydroxide with the EPh Basic. Ex 1 through Ex 4 include the use of the 50% Aqueous Sodium Hydroxide with the EPh Basic, while Ex 5 through Ex 12 include the use of a dehydration step and the 50% Aqueous Sodium Hydroxide with the EPh Basic.

For Table 2, the Evaporator Temperature (Evap Temp, ° C.) is the temperature set for both the lower zone and the upper zone of the internal evaporative area 102; the Evaporator Pressure (Evap Press, Torr) is the pressure inside internal evaporative area 102; the Feed Weight (Feed Wt.) is the weight of the EPh Basic feed in the 2-liter jacketed feed vessel 104; the grams of 50% NaOH per gram Feed (g 50% NaOH per g Feed) is the weight of the 50% Aqueous Sodium Hydroxide added per gram of the EPh Basic feed into the internal evaporative area 102; the weight percent NaOH (wt. % NaOH in Feed) is the wt. % of NaOH present in the EPh Basic feed into the internal evaporative area 102, where the wt. % of NaOH is measured as described in the Base Concentration Measurement section below.

In Table 2, “Dehy” indicates the use of a dehydration step “Via WFE” or “Via Roto.” For the dehydration step “Via WFE” heat the upper zone and the lower zone of the internal evaporative area 102 to 60° C. under a vacuum of 13.3 Pa to 133.3 Pa (0.1 Torr to 1 Torr). Measure the amount of water present in the EPh Basic feed in the 2-liter jacketed feed vessel 104 using Karl Fischer analysis, as described herein. Add the EPh Basic feed to the internal evaporative area 102 to allow for dehydration, removing only the water but taking no overhead product from the evaporator section 112. Hold the EPh Basic feed to the internal evaporative area 102 for a time sufficient to reduce the amount of water present in the heavies fraction collected in the round bottom flask 120 to be less than 0.05 wt. % as measured using Karl Fischer analysis.

For the dehydration step “Via Roto” heat the EPh Basic feed in the round bottom flask of a Rotary Evaporator to a temperature of 60° C. under a vacuum of 13.3 KPa to 1.3 KPa (100 Torr to 10 Torr) until the EPh Basic feed has an amount of water of less than 0.05 wt. % as measured using Karl Fischer analysis.

The resulting dehydrated EPh Basic is then separated using the operating conditions and settings of the rolled film evaporator 100 seen in Table 2. Table 3 provides the GC analysis results for the Examples and Comparative Examples of Table 2.

TABLE 2

| | Evap Temp | Press | Feed | g 50% NaOH | Wt. % NaOH | | Add | Over- | Heavies, | Wt. % | Wt. % NaOH |
|---|---|---|---|---|---|---|---|---|---|---|---|
| Ex/CE | ° C. | Pa | Wt., g | per g Feed | in Feed | Dehy. | Time, min. | head, g | g | Recov | Heavies |
| CE A | 140 | 13.3 | 1231.16 | 0 | 1.29 | No | 35 | 681.09 | 509.57 | 55.3 | 2.89 |
| CE B | 140 | 13.3 | 1169.9 | 0 | 1.29 | No | 110 | 878.44 | 259.56 | 75.1 | 5.18 |
| CE C | 125 | 13.3 | 1157.44 | 0 | 1.29 | No | 30 | 376.55 | 741.65 | 32.5 | 1.91 |
| CE D | 125 | 13.3 | 1132.86 | 0 | 1.29 | No | 125 | 684.11 | 405.44 | 60.4 | 3.26 |
| CE E | 140 | 26.6 | 1293.28 | 0.0072 | 1.57 | No | 40 | 762.19 | 462.89 | 58.9 | 3.83 |
| Ex 1 | 140 | 13.3-426.6 | 1221.45 | 0.0420 | 3.14 | No | 33 | 202.58 | 975.02 | 16.6 | 3.77 |
| Ex 2 | 125 | 13.3-53.3 | 1195.21 | 0.0422 | 3.20 | No | 136 | 534.91 | 456.38 | 44.8 | 5.79 |
| Ex 3 | 125 | 13.3 | 1228.34 | 0.0251 | 2.28 | No | 140 | 702.6 | 343.95 | 57.2 | 5.33 |
| Ex 4 | 125 | 13.3 | 1216.58 | 0.0345 | 2.91 | No | 125 | 650.42 | 399.85 | 53.5 | 6.26 |
| Ex 5 | 140 | 13.3 | 1143.27 | 0.0300 | 2.69 | Via | 38 | 637.02 | 504.23 | 55.7 | 6.07 |
| | | | | | | WFE | | | | | |
| Ex 6 | 140 | 13.3 | 1172.78 | 0.0300 | 2.69 | Via | 34 | 645.83 | 411.33 | 55.1 | 5.98 |
| | | | | | | Roto | | | | | |
| Ex 7 | 160 | 666.6 | 1121.65 | 0.0288 | 2.57 | Via | 132 | 264.25 | 866.5 | 23.6 | 3.36 |
| | | | | | | WFE | | | | | |
| Ex 8 | 170 | 666.6 | 1112.33 | 0.0293 | 2.47 | Via | 115 | 621.0 | 416.86 | 55.8 | 5.59 |
| | | | | | | WFE | | | | | |
| Ex 9 | 180 | 666.6 | 1139.64 | 0.0300 | 2.70 | Via | 37 | 638.66 | 484.41 | 56.0 | 6.15 |
| | | | | | | WFE | | | | | |
| Ex 10 | 190 | 666.6 | 1168.28 | 0.0300 | 2.69 | Via | 35 | 765.9 | 372.4 | 65.6 | 7.82 |
| | | | | | | WFE | | | | | |
| Ex 11 | 150 | 13.3 | 1226.68 | 0.0302 | 2.68 | Via | 38 | 741.0 | 261.97 | 60.4 | 6.78 |
| | | | | | | WFE | | | | | |
| Ex 12 | 210 | 13.3 | 1105.79 | 0.0302 | 2.68 | Via | 30 | 866.6 | 87.7 | 78.4 | 12.41 |
| | | | | | | WFE | | | | | |

TABLE 3
GC Analysis Results

| | GC Area % | | | | | | | | | |
|---|---|---|---|---|---|---|---|---|---|---|
| | | | | | | 26.5 | | | | 30.8 |
| Ret. Time (min) | 18.4 | 21.2 | 22.2 | 23.4 | 25.600 | Isomer of | 26.9 | 27.3 | 29.4 | Isomer of |
| Identification | EPh | 2-HEPA | 4-HEPA | DiEPh | TriEPh | TetraEPh | Highers 2 | Highers 1 | TetraEPh | TetraEPh |
| MW | 138.15 | 138.15 | 138.15 | 182.19 | 226.23 | 270.27 | 270.27 | 270.27 | 270.27 | 270.27 |
| Sample | | | | | | | | | | |
| DiEPh Std. | 16.57 | | | 65.86 | 6.960 | | 1.87 | 6.44 | 1.06 | |
| Comp. wt. % | 13.44 | 0.0149 | 0 | 64.4 | 7.720 | | 1.77 | 7.77 | | |
| EPh Basic | 14.211 | 9.743 | 2.294 | 59.365 | 7.664 | 0.211 | 1.713 | 3.621 | 1.188 | |
| CE A - OH | 23.5 | 3.68 | 0.41 | 70.8 | 1.610 | | | | | |
| CE A - BT | 3.29 | 17.3 | 4.56 | 46.39 | 14.910 | 0.46 | 3.49 | 6.9 | 2.7 | |
| CE B - OH | 18.116 | 6.108 | 0.697 | 69.57 | 3.487 | | 0.511 | 1.511 | | |
| CE B - BT | 3.914 | 17.359 | 5.514 | 38.205 | 17.513 | 0.59 | 4.485 | 8.245 | 3.607 | 0.6 |
| CE C - OH | 35.908 | 2.267 | 0.224 | 60.709 | 0.893 | | 0.098 | 0.269 | | |
| CE C - BT | 3.481 | 13.566 | 3.329 | 58.194 | 10.999 | 0.318 | 2.501 | 5.388 | 1.907 | 0.318 |
| CE D - OH | 21.841 | 4.122 | 0.373 | 71.505 | 1.608 | | 0.189 | 0.363 | | |
| CE D - BT | 4.599 | 15.069 | 3.928 | 50.12 | 13.387 | 0.373 | 3.107 | 6.724 | 2.305 | 0.387 |
| CE E - OH | 23.528 | 2.644 | 0.351 | 71.041 | 1.569 | | 0.227 | 0.64 | | |
| CE E - BT | 1.534 | 18.527 | 4.667 | 44.555 | 15.374 | 0.476 | 3.613 | 7.451 | 2.887 | 0.916 |
| Ex 1 - OH | 52.745 | 0.136 | 0.155 | 46.434 | 0.458 | | 0.082 | | | |
| Ex 1 - BT | 5.026 | 11.971 | 2.839 | 62.09 | 9.474 | 0.271 | 2.126 | 4.582 | 1.619 | |
| Ex 2 - OH | 29.727 | 0.075 | | 69.787 | 0.410 | | | | | |
| Ex 2 - BT | 0.96 | 17.354 | 4.038 | 52.963 | 13.288 | 0.371 | 2.975 | 6.144 | 1.906 | |
| Ex 3 - OH | 24.008 | 0.514 | | 74.773 | 0.705 | | | | | |
| Ex 3 - BT | 0.54 | 22.86 | 5.4 | 37.802 | 17.292 | 0.51 | 3.912 | 8.129 | 3.028 | 0.525 |
| Ex 4 - OH | 25.536 | 0.067 | | 73.923 | 0.475 | | | | | |
| Ex 4 - BT | 0.377 | 21.849 | 5.127 | 41.931 | 16.533 | 0.477 | 3.743 | 7,293 | 2.135 | 0.535 |
| Ex 5 - OH | 24.78 | 0.232 | | 73.306 | 0.968 | | 0.183 | 0.53 | | |
| Ex 5 - BT | 3.421 | 19.2 | 4.472 | 49.816 | 14.281 | 0.412 | 3.135 | 4 | 1.987 | |
| Ex 6 - OH | 24.658 | 0.382 | | 73.077 | 1.090 | | 0.212 | 0.581 | | |
| Ex 6 - BT | 0.0585 | 22.043 | 5.166 | 41.595 | 16.193 | 0.487 | 3.677 | 7 | 2.784 | |
| Ex 7 - OH | 48.183 | 0.138 | | 51.588 | 0.090 | | | | | |
| Ex 7 - BT | 4.081 | 11.622 | 2.739 | 63.862 | 9.309 | 0.259 | 2.087 | 5 | 1.49 | |
| Ex 8 - OH | 25.188 | 0.302 | | 66.473 | 7.080 | | 0.141 | 0.816 | | |
| Ex 8 - BT | 2.339 | 18.768 | 4.383 | 46.953 | 14.278 | 0.412 | 3.147 | 6.471 | 2.815 | 0.434 |
| Ex 9 - OH | 23.502 | 0.263 | | 73.731 | 1.490 | | 0.303 | 0.712 | | |
| Ex 9 - BT | 7.094 | 13.776 | 3.222 | 58.007 | 10.464 | 0.304 | 2.308 | 3.064 | 1.76 | |
| Ex 10 - OH | 20.637 | 0.389 | | 74.642 | 2.234 | | 0.485 | 1.613 | | |
| Ex 10 - BT | 6.391 | 16.657 | 3.907 | 50.754 | 12.032 | 0.372 | 2.676 | 5.336 | 1.866 | |
| Ex 11 - OH | 22.417 | 0.314 | | 74.925 | 1.335 | | 0.229 | 0.78 | | |
| Ex 11 - BT | 7.46 | 9.5 | 2.173 | 63.883 | 8.798 | 0.197 | 2 | 4.736 | 1.245 | |
| Ex 12 - OH | 17.391 | 0.881 | 0.155 | 71.76 | 4.920 | | 1.396 | 3.495 | | |
| Ex 12 - BT | 9.084 | 5.603 | 1.304 | 54.884 | 13.321 | | 4.795 | 8.140 | 2.595 | |

OH—Overhead Product; BT—Bottoms Product. For the above Table EG has a MW of 62.04 and a retention time of 10.7 min.; Phenol has a MW of 94.11 and a retention time of 12.2 min.; and DEG has a MW of 106.08 and a retention time of 18.4 min.

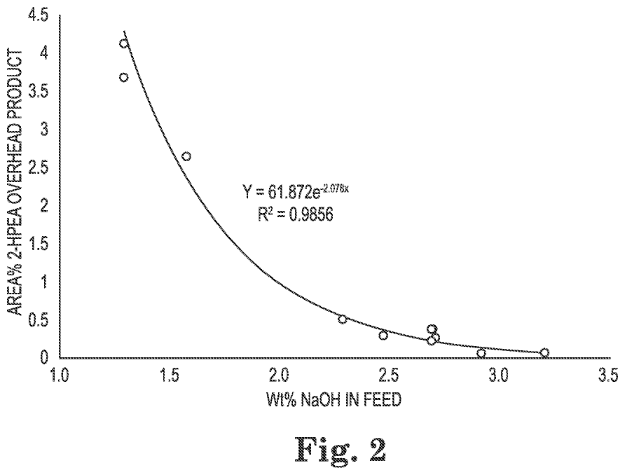

FIG. 2 shows the relationship of the GC area percent data for the 2-HPEA in the overhead product (exiting the evaporator section 112) as a function of the weight percent of NaOH present in the test sample of the EPh Basic (present in the feed vessel 104). The data points plotted in FIG. 2, moving from left to right across the x-axis, are for CE A, CE B, CE E, Ex 3, Ex 8, Ex 5, Ex 7, Ex 11, Ex 4 and Ex 2.

As illustrated by FIG. 2, thin film evaporation of EPh basic that has been charged with additional sodium hydroxide was able to substantially reduce the 2-HPEA content in the EPh basic to below 1 weight percent based on the total weight of the EPh basic.

1. A method for isolating dialkylene phenolic glycol ether from a mixture that includes dialkylene phenolic glycol ether and glycosylated phenol impurities, the method comprising:
adding a source of an alkali metal to the mixture to form a phenolic glycol product having an alkali phenolic salt formed from a reaction between the alkali metal and the glycosylated phenol impurities; and
separating the dialkylene phenolic glycol ether from the alkali phenolic salt in the phenolic glycol product through a thin film evaporation process to produce a dialkylene phenolic glycol ether product having less than 1 wt. % of the glycosylated phenol impurities based on the total weight of the dialkylene phenolic glycol ether product.

2. The method of claim 1, wherein the mixture further includes water, wherein the phenolic glycol product undergoes a dehydrating step to remove at least a portion of the water from the phenolic glycol product prior to separating the dialkylene phenolic glycol ether from the alkali phenolic salt in the phenolic glycol product.

3. The method of claim 1, wherein the alkali metal is selected from the group consisting of sodium and potassium.

4. The method of claim 3, wherein the source for the alkali metal is an alkali hydride.

5. The method of claim 4, wherein the alkali hydride is selected from the group consisting of sodium hydride and potassium hydride.

6. The method of claim 3, wherein the source for the alkali metal is an alkali hydroxide.

7. The method of claim 6, wherein the alkali hydroxide is selected from the group consisting of sodium hydroxide and potassium hydroxide.

8. The method of claim 7, wherein the source for the alkali metal is an aqueous solution of the alkali hydroxide.

9. The method of claim 8, wherein the aqueous solution of the alkali hydroxide is an aqueous sodium hydroxide solution having an amount of sodium hydroxide up to 50 percent by weight of the aqueous sodium hydroxide solution.

10. The method of claim 6, wherein adding the source of the alkali metal to the mixture includes adding 1.6 weight percent (wt. %) to 5 wt. % of the alkali metal hydroxide to the mixture, wherein the wt. % is based on the total weight of the phenolic glycol product.

11. The method of claim 6, wherein adding the source of the alkali metal to the mixture includes adding 2 weight percent (wt. %) to 3.5 wt. % of the alkali metal hydroxide to the mixture, wherein the wt. % is based on the total weight of the phenolic glycol product.

12. The method of claim 1, wherein the glycosylated phenol impurities have a boiling point at a predetermined pressure that are approximately equivalent to the boiling point of the dialkylene phenolic glycol ether at the predetermined pressure.

13. The method of claim 1, wherein the dialkylene phenolic glycol ether is diethylene glycol phenyl ether and the glycosylated phenol impurities include 2-hydroxyphenylethanol and 4-hydroxyphenylethanol.

14. The method of claim 1, wherein the dialkylene phenolic glycol ether is dipropylene glycol phenyl ether and the glycosylated phenol impurities include 2-hydroxyphenylpropanol and 4-hydroxyphenylpropanol.

15. A phenolic glycol product, comprising:
dialkylene phenolic glycol ether;
water;
glycosylated phenol impurities;
a source of alkali metal; and
an alkali phenolic salt formed from a reaction between the alkali metal and the glycosylated phenol impurities, wherein the phenolic glycol product has less than 1 wt. % of the glycosylated phenol impurities based on the total weight of the dialkylene phenolic glycol ether product.

16. The phenolic glycol product of claim 15, wherein the source of alkali metal is selected from the group consisting of sodium, sodium hydroxide, sodium hydride, potassium, potassium hydroxide and potassium hydride.

17. The phenolic glycol product of claim 15, wherein the phenolic glycol product includes 1.6 weight percent (wt. %) to 5 wt. % of the source of alkali metal, the wt. % based on the total weight of the phenolic glycol product.

18. The phenolic glycol product of claim 15, wherein the phenolic glycol product includes 2 weight percent (wt. %) to 3.5 wt. % of the source of alkali metal, the wt. % based on the total weight of the phenolic glycol product.

19. The phenolic glycol product of claim 15, wherein the dialkylene phenolic glycol ether is diethylene glycol phenyl ether and the glycosylated phenol impurities include 2-hydroxyphenylethanol and 4-hydroxyphenylethanol.

20. The phenolic glycol product of claim 15, wherein the dialkylene phenolic glycol ether is dipropylene glycol phenyl ether and the glycosylated phenol impurities include 2-hydroxyphenylpropanol and 4-hydroxyphenylpropanol.
